(19) United States
(12) Patent Application Publication
(10) Pub. No.: US 2012/0046321 A1
(43) Pub. Date: Feb. 23, 2012

(54) METHODS OF TREATING ATRIAL FIBRILLATION WITH P38 INHIBITOR COMPOUNDS

(75) Inventors: Jeff Olgin, Larkspur, CA (US); Susan Eisenberg, Orinda, CA (US); Lawrence M. Blatt, San Francisco, CA (US); Scott D. Seiwert, Pacifica, CA (US); Karl Kossen, Brisbane, CA (US)

(73) Assignee: INTERMUNE, INC., Brisbane, CA (US)

(21) Appl. No.: 13/244,707

(22) Filed: Sep. 26, 2011

Related U.S. Application Data
(63) Continuation of application No. 12/091,161, filed on Oct. 9, 2009.
(60) Provisional application No. 60/732,676, filed on Nov. 1, 2005.

(51) Int. Cl. A61K 31/4418; A61P 9/06; A61P 9/00

(52) U.S. Cl. 514/345

(57) ABSTRACT

The invention disclosed herein relates generally to compounds and methods useful in treating or preventing atrial fibrillation (AF).

### CROSS-REFERENCE TO RELATED APPLICATIONS

This application claims the benefit of U.S. Provisional Patent Application No. 60/732,676, filed Nov. 1, 2005, which is incorporated herein by reference in its entirety for all purposes.

### FIELD OF THE INVENTION

This invention relates generally to compounds and methods useful in treating or preventing atrial fibrillation.

### BACKGROUND OF THE INVENTION

Atrial fibrillation (AF or A-fib) is one of the most common arrhythmia and one of the leading causes of cardiovascular disease-related morbidity in the world. It is estimated that between 2 and 3 million Americans suffer from AF. In normal sinus rhythm, the atria (the upper chambers of the heart) contract, the valves open, and blood fills the ventricles (the lower chambers). The ventricles then contract to complete the organized cycle of each heart beat. AF involves an abnormality of electrical impulse formation and conduction that originates in the atria causing the atria to quiver or fibrillate instead of beat effectively. The heart normally contracts (beats) 60 to 80 times per minute at rest. In AF, the atria fibrillate as many as 300-600 times/minute. During AF, the blood is not able to empty efficiently from the atria into the ventricles with each heart beat. Blood may then pool and become stagnant in the atria, creating a site for blood clot formation. Such clot formation may become a primary source of stroke in patients with AF. Other complications of AF include congestive heart failure and cardiomyopathy.

AF may be chronic or paroxysmal. In chronic or persistent AF, the atria fibrillate all of the time. In paroxysmal AF, the patient experiences intermittent episodes of AF that occur with varying frequency and last for a variable period of time before spontaneously reverting to normal between episodes

AF may occur in patients with any type of underlying structural heart abnormality, such as coronary artery disease, valvular heart disease, congenital heart disease, and cardiomyopathies of various kinds, thereby complicating patient management and therapy. In addition, AF occurs in as many as 50% of patients undergoing cardiac operations. Further, AF may sometimes occur in patients with no known underlying structural abnormalities (lone AF) or in patients with lung disease or hormonal or metabolic disorders. AF may occur at any age, but its prevalence tends to increase with age and effects men slightly more often than women. The occurrence of AF may exacerbate other disorders, for example, myocardial ischemia or congestive heart failure.

Many conditions have been associated with AF, including thyroid disorders, valve disease, hypertension, sick sinus syndrome, pericarditis, lung disease, and congenital heart defects. Patients with chronic AF may suffer from symptomatic tachycardia or low cardiac output, have a risk of thromboembolic complications, and are at risk for death.

Several approaches are used to treat and prevent abnormal beating. Non-surgical treatments are sometimes effective in treating AF. Several drugs are known, for example, digoxin, beta blockers (atenolol, metoprolol, propranolol), amiodarone, disopyramide, calcium antagonists (verapamil, diltiazam), sotalol, flecamide, procainamide, quinidine and propafenone, but may have significant and/or intolerable side effects, including pro-arrhythmic effects, that is, causing other abnormal heart rhythms, and thus, are not ideal for treatment of acute fibrillation or diseases of the heart muscle or coronary arteries. Moreover, some drugs have hemodynamic effects that may play a role in treating AF, but that may limit their use in clinical settings. Finally, these drugs may not be effective long term as many patients develop a recurrence of AF. Electrical cardioversion (alone or in combination with anti-arrhythmic therapy) may be used to restore normal sinus rhythm with an electric shock, however, high recurrences of AF have been reported.

A number of invasive surgical procedures are used for treatment of AF. Invasive procedures involving direct visualization of the tissues include the Maze procedure, in which the atria are surgically dissected and then repaired. In the Maze procedure, for example, ectopic re-entry pathways of the atria are interrupted by the scar tissue formed using a scalpel or the like. The pattern of scar tissue then prevents the recirculating electrical signals that result in AF.

Ablation is sometimes used to terminate AF by introducing a catheter into the heart and directing energy at specific areas of heart tissue. Radiofrequency energy has been used to terminate AF by introducing a catheter into the heart and directing a burst of radiofrequency energy to specific areas of the heart to destroy tissue that triggers abnormal electrical signals or to block abnormal electrical pathways. In addition, surgery may be used to disrupt electrical pathways that generate AF. Atrial pacemakers may be implanted under the skin to regulate the heart rhythm. Nonetheless, there is still a need for non-invasive treatments of AF that have long-term efficacy.

As discussed above, AF has traditionally been treated with antiarrhythmic drugs, with their accompanying proarrhythmia risks. Nattel S, Newer, Am Heart J. 1995; 130:1094-106; Roden D M; Am J Cardiol. 1998; 82:491-571; Nattel S., Cardiovasc Res. 2002; 54:347-60. Recently, pharmacologic therapy targeted at the underlying substrate has been investigated. Kumagai K, et al., J Am Coll Cardiol. 2003; 41:2197-204; Li D, et al., Circulation. 2001; 104:2608-14. While ACE inhibitors and AT1-R antagonists are promising and have been shown to be effective in attenuating atrial structural remodeling, these drugs have hemodynamic effects and the perturbation in hemodynamics, as observed in canine models of AF (Kumagai K, et al., J Am Coll Cardiol. 2003; 41:2197-204; Li D, et al., Circulation. 2001; 104:2608-14), may play a role in attenuating atrial remodeling. In certain clinical settings, the hemodynamic effects of these classes of drugs may potentially limit their use. Thus, there is a need for pharmacologic therapy for AF, and particularly for therapy that substantially lacks hemodynamic effects.

### SUMMARY OF THE INVENTION

Disclosed herein are compositions and methods for the treatment or prevention of atrial fibrillation (AF).

Accordingly, some embodiments provide a method for treating AF, wherein the methods comprise administering to a subject in need of such treatment a therapeutically effective amount of a p38 inhibitor compound. In some embodiments, the method further comprises identifying a subject suffering from or at risk of developing atrial fibrillation. Preferably, the subject is a human. In some embodiments the therapeutically effective amount of the p38 inhibitor compound prevents, suppresses, inhibits, and/or terminates the fibrillation. In some embodiments, the therapeutically effective amount of the p38 inhibitor compound restores normal sinus rhythm.

Other embodiments provide a method of treating (e.g. preventing) arrhythmia in a subject in need of such treatment, comprising administering a therapeutically effective amount of a p38 inhibitor compound to the subject. In some embodiments, the arrhythmia is atrial fibrillation. In some embodiments, the method further includes identifying a subject suffering from an arrhythmia.

Some embodiments provide a method of preventing atrial fibrillation in a subject in need of such prevention (e.g. a subject having a heart disorder) comprising administering a therapeutically effective amount of a p38 inhibitor compound to the subject. In some embodiments, the method further includes identifying a subject suffering from a heart disorder.

Other embodiments provide a pharmaceutical composition to treat (e.g. suppress) atrial fibrillation comprising an effective treating or suppressing amount of a p38 inhibitor compound.

In some embodiments, the p38 inhibitor compound is a low-potency p38 inhibitor compound. In some embodiments, the low-potency p38 inhibitor compound exhibits an IC₅₀ in the range of about 100 μM to about 1000 μM for inhibition of p38 MAPK. In other embodiments, the p38 inhibitor compound binds to the ATP binding site of the p38 MAPK thereby decreasing the activity of the p38 MAPK relative to the activity of the p38 MAPK in the absence of inhibitor. In other embodiments, the p38 inhibitor compound competitively binds to the ATP binding site of the p38 thereby decreasing the activity of the p38 MAPK relative to the activity of the p38 MAPK in the absence of inhibitor.

In some embodiments, the therapeutically effective amount produces a blood or serum or other bodily fluid concentration that is less than an IC₃₀ for inhibition of p38 MAPK. In some embodiments, the therapeutically effective amount is less than 50% of an amount that causes an undesirable side effect in the subject. In some embodiments, the p38 inhibitor substantially lacks hemodynamic effects.

In some embodiments, the p38 inhibitor compound is pirfenidone. In some embodiments, the p38 inhibitor compound is selected from Compounds 1 to 23 in Table 1 below. In some embodiments, the compositions comprise a p38 inhibitor compound in combination with a pharmaceutically acceptable carrier. In some embodiments, the compositions are formulated for oral administration.

In some embodiments, the methods comprise administering a tablet or capsule, wherein the tablet or capsule comprises the p38 inhibitor compound. In some embodiments, the methods comprise administering one or more of the tablets or capsules to the subject one or more times per day. In some embodiments, the methods comprise administering one or more of the capsules to the subject twice per day. In some embodiments, the methods comprise administering one or more capsules to the subject three times per day.

In some embodiments, the p38 inhibitor compound is provided in a dose of from about 100 to about 400 milligrams. In some embodiments, the method comprises administering the p38 inhibitor compound such that the daily intake of the p38 inhibitor compound is from about 800 to about 4000 mg/day. In some embodiments, the method comprises administering the p38 inhibitor compound such that the daily intake of said p38 inhibitor compound is about 1200 mg/day or higher.

### BRIEF DESCRIPTION OF THE DRAWINGS

FIG. 1 is a bar graph showing left atrial (LA) area measurements at baseline and percent change from baseline over the 3-week VTP period in the CHF and CHF+PFD groups.

FIG. 2 is a bar graph showing AF inducibility for normal, CHF, and CHF+PFD groups.

FIGS. 3A-3D are bar graphs showing effective refractory period (ERP) (FIGS. 3A and 3B) and conduction velocity (CV) (FIGS. 3C and 3D) findings among each of the study groups at 3 pacing BCLs.

FIGS. 4A-4L are representative isochronal activation maps (from each of the 4 individual atrial epicardial plaques) at a pacing BCL of 300 ms. Plaque activation time color map: red=earliest, blue=latest.

FIGS. 5A-5D are bar graphs showing absolute conduction heterogeneity (P95-5) (FIGS. 5A and 5B) and conduction heterogeneity index (P95-5/P50) (FIGS. 5C and 5D) findings for the atria at 3 pacing BCLs.

FIGS. 6A-6I are representative LA sections stained with Sirius red at magnifications of 50×, 100×, and 400×.

FIG. 7 is a bar graph sowing percent left atrial fibrosis.

FIGS. 8A-8I are representative Western immunoblot finding for fibrosis and inflammation mediators: TGF-β1, total ERK ½ (42- and 44 k-Da isoforms), total JNK (46- and 54-kDa isoforms), total p-38, TIMP-4, MMP-9 (active form, 88 kDa), TNF-α, IL-6, IL-10.

FIGS. 9A-9D are representative immunofluorescent Cx40 and Cx43 distribution findings from LA specimens of CHF and CHF+PFD canines.

### DETAILED DESCRIPTION OF THE PREFERRED EMBODIMENTS

It has now been discovered that a high therapeutic effect in treating AF may be achieved using a p38 kinase inhibitor compound.

Accordingly, in one embodiment methods of treating or preventing AF are provided, the methods comprising the use of a p38 inhibitor compound. Examples of p38 inhibitor compounds useful in the invention are described herein and discussed more fully below.

The methods may include identifying a subject at risk for or suffering from AF or a condition associated with AF and administering a compound to the subject in an effective amount to treat or prevent the condition. The term “at risk for or suffering from” as used herein, refers to subjects suffering from chronic or paroxysmal AF or a condition associated with AF, including subjects currently experiencing an AF episode and those not currently experiencing an AF episode, as well as subjects who have not been diagnosed with AF, but who have been identified as being at risk for developing AF. Methods for identifying a subject at risk for or suffering from AF or a condition associated with AF are known in the art. Thus, in some embodiments, the compound is administered to a patient currently experiencing an AF. In another embodiment, the compound is administered to a patient diagnosed with AF but not currently experiencing an AF episode. In still another embodiment, the compound is administered to a patient who has not been diagnosed with AF, but who has been identified as being at risk for developing AF. Risk factors of AF are well known in the art, and include, but are not limited to, increased age, high blood pressure, heart failure of almost any cause, congenital heart disease, coronary heart disease, including heart attack or myocardial infarction, abnormal heart muscle function, including congestive heart failure, disease of the mitral valve between the left and right ventricles, pericarditis, hyperthyroidism, overdose of thyroid medication, low amounts of oxygen in the blood, chronic lung diseases, including emphysema, asthma, or chronic obstructive pulmonary disease (COPD), pulmonary embolism, physical or psychological stress, excessive alcohol intake, stimulant drug use, such as cocaine or decongestants, and recent heart or lung surgery.

In an embodiment, the compound used in the methods described herein is a p38 inhibitor compound. In some embodiments, the compound is a low potency p38 inhibitor that exhibits, for example, an IC₅₀ in the range of about 100 μM to about 1000 μM, or about 200 μM to about 800 μM for inhibition of a p38 MAP kinase (MAPK). In some embodiments, the effective amount produces a blood or serum or another bodily fluid concentration that is less than an IC₃₀ or an IC₂₀ or an IC₁₀ for inhibition of p38 MAPK by the compound. In some embodiment, the In some embodiments, the effective amount is about 70% or less, or about 50%, of an amount that causes an undesirable side effect in the subject, such as, but not limited to, drowsiness, gastrointestinal upset, and photosensitivity rash. The compound used for the treatment or prevention may be pirfenidone or a compound of Genera Ia-c, Subgenera II-V and/or Genus VI as described below. In a preferred embodiment, the compound substantially lacks hemodynamic effects.

A preferred subject is a mammal. A mammal may include any mammal. As a non-limiting example, preferred mammals include cattle, pigs, sheep, goats, horses, camels, buffalo, cats, dogs, rats, mice, and humans. A highly preferred subject mammal is a human. The compound(s) may be administered to the subject via any drug delivery route known in the art, including for example, but not limited to, oral, ocular, rectal, buccal, topical, nasal, ophthalmic, subcutaneous, intramuscular, intravenous (bolus and infusion), intracerebral, transdermal, and pulmonary.

The terms “therapeutically effective amount” and “prophylactically effective amount,” as used herein, refer to an amount of a compound sufficient to treat (e.g. ameliorate or prevent) the identified disease or condition, or to exhibit a detectable therapeutic, prophylactic, and/or inhibitory effect. For example, the effect may be restoration of normal sinus rhythm, reduction of AF burden, either in time spent in AF or in duration of AF episodes, reduction in atrial fibrosis, suppression of AF, termination of AF, inhibition of AF, prevention of recurrence of AF, prevention of developing AF, and the like. The effect may be detected by any means known in the art. The precise effective amount for a subject will depend upon the subject's body weight, size, and health; the nature and extent of the condition; and the therapeutic or combination of therapeutics selected for administration. Therapeutically and prophylactically effective amounts for a given situation may be determined by routine experimentation that is within the skill and judgment of the clinician. In some embodiments, the effective amount of the compound of the embodiments produces a blood or serum or another bodily fluid concentration that is less than an IC₃₀, IC₂₀ or IC₁₀ for inhibition of a p38 MAPK.

For any compound, the therapeutically or prophylactically effective amount may be estimated initially either in cell culture assays or in animal models, usually rats, mice, rabbits, dogs, or pigs. The animal model may also be used to determine the appropriate concentration range and route of administration. Such information may then be used to determine useful doses and routes for administration in humans.

Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it may be expressed as the ratio, ED₅₀/LD₅₀. Pharmaceutical compositions that exhibit large therapeutic indices are preferred. However, the pharmaceutical compositions that exhibit narrow therapeutic indices are also within the scope of the embodiments. The data obtained from cell culture assays and animal studies may be used in formulating a range of dosage for human use. The dosage contained in such compositions is preferably within a range of circulating concentrations that include an ED₅₀ with little or no toxicity. The dosage may vary within this range depending upon the dosage form employed, sensitivity of the patient, and the route of administration.

More specifically, the maximum plasma concentrations (Cₘₐₓ) may range from about 65 μM to about 115 μM, or about 75 μM to about 105 μM, or about 85 μM to about 95 μM, or about 85 μM to about 90 μM depending upon the route of administration. In general the dose will be in the range of about 100 mg/day to about 10 g/day, or about 200 mg to about 5 g/day, or about 400 mg to about 3 g/day, or about 500 mg to about 2 g/day, in single, divided, or continuous doses for a patient weighing between about 40 to about 100 kg (which dose may be adjusted for patients above or below this weight range, particularly children under 40 kg). Generally the dose will be in the range of about 25 mg/kg to about 300 mg/kg of body weight per day. In one embodiment, the p38 inhibitor compound is administered to the subject in a unit dosage form comprising about 100 to about 400 mg of the p38 inhibitor compound per dose. The dosing may be once, or twice or three times daily, with one or more units per intake. According to one embodiment, the total daily intake is at least about 1200 mg of the p38 inhibitor compound.

The exact dosage will typically be determined by the practitioner, in light of factors related to the subject that requires treatment. Dosage and administration are generally adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

It will be appreciated that treatment as described herein includes preventing a disease, ameliorating symptoms, slowing disease progression, reversing damage, or curing a disease.

In one aspect, treating AF results in an increase in average survival time of a population of treated subjects in comparison to a population of untreated subjects. Preferably, the average survival time is increased by more than about 30 days; more preferably, by more than about 60 days; more preferably, by more than about 90 days; and even more preferably by more than about 120 days. An increase in survival time of a population may be measured by any reproducible means. In a preferred aspect, an increase in average survival time of a population may be measured, for example, by calculating for a population the average length of survival following initiation of treatment with an active compound. In an another preferred aspect, an increase in average survival time of a population may also be measured, for example, by calculating for a population the average length of survival following completion of a first round of treatment with an active compound.

In another aspect, treating AF results in a decrease in the mortality rate of a population of treated subjects in comparison to a population of subjects receiving carrier alone. In another aspect, treating AF results in a decrease in the mortality rate of a population of treated subjects in comparison to an untreated population. In a further aspect, treating AF results a decrease in the mortality rate of a population of treated subjects in comparison to a population receiving monotherapy with a drug that is not a compound of the embodiments, or a pharmaceutically acceptable salt, metabolite, analog or derivative thereof. Preferably, the mortality rate is decreased by more than about 2%; more preferably, by more than about 5%; more preferably, by more than about 10%; and most preferably, by more than about 25%. In a preferred aspect, a decrease in the mortality rate of a population of treated subjects may be measured by any reproducible means. In another preferred aspect, a decrease in the mortality rate of a population may be measured, for example, by calculating for a population the average number of disease-related deaths per unit time following initiation of treatment with an active compound. In another preferred aspect, a decrease in the mortality rate of a population may also be measured, for example, by calculating for a population the average number of disease related deaths per unit time following completion of a first round of treatment with an active compound.

In another aspect, treating AF results in a decrease in AF burden, either time spent in AF or duration of AF episodes. Preferably, after treatment, the AF burden is reduced by at least about 5% relative to the AF burden prior to treatment; more preferably, AF burden is reduced by at least about 10%; more preferably, reduced by at least about 20%; more preferably, reduced by at least about 30%; more preferably, reduced by at least about 40%; more preferably, reduced by at least about 50%; even more preferably, reduced by at least 60%; and most preferably, reduced by at least about 75%. AF burden may be measured by any reproducible means of measurement. In a preferred aspect, AF burden is measured using an electronic recording device.

In another aspect, treating AF and/or administration of a p38 inhibitor results in a reduction of ERK expression relative to ERK expression in the absence of p38 inhibitor. In some embodiments, after treatment or administration, ERK expression is reduced by at least about 5%; at least about 10%; at least about 20%; at least about 30%; at least about 40%; at least about 50%; at least about 60%; or at least about 75%. ERK expression may be measured by any reproducible means of measurement.

In another aspect, treating AF and/or administration of a p38 inhibitor results in a reduction in p38 expression relative to p38 expression in the absence of p38 inhibitor. In some embodiments, after treatment or administration, p38 expression is reduced by at least about 5%; at least about 10%; at least about 20%; at least about 30%; at least about 40%; at least about 50%; at least about 60%; or at least about 75%. Reduction in p38 expression may be measured by any reproducible means of measurement.

In another aspect, treating AF and/or administration of a p38 inhibitor results in a decrease in c-Jun expression relative to c-Jun expression in the absence of p38 inhibitor. In some embodiments, after treatment or administration, c-Jun expression is reduced by at least about 5%; at least about 10%; at least about 20%; at least about 30%; at least about 40%; at least about 50%; at least about 60%; or at least about 75%. Reduction in c-Jun expression may be measured by any reproducible means of measurement.

In another aspect, treating AF and/or administration of a p38 inhibitor results in a decrease in TGF-β1 expression relative to TGF-β1 expression in the absence of p38 inhibitor. In some embodiments, after treatment or administration, TGF-β1 expression is reduced by at least about 5%; at least about 10%; at least about 20%; at least about 30%; at least about 40%; at least about 50%; at least about 60%; or at least about 75%. Reduction in TGF-β1 expression may be measured by any reproducible means of measurement.

In some embodiments, p38 inhibitors useful in the methods disclosed herein reduce the expression of any or all of ERK, p38, Jun and TGF-β1. That is, in some embodiments, the expression of ERK, p38, Jun and TGF-β1 are all reduced following administration of a p38 inhibitor compound relative to the expression of these proteins in the absence of p38 inhibitor administration and/or relative to the expression of these proteins prior to administration of the p38 inhibitor compound. In some embodiments, the expression of only some of these proteins is reduced following administration of a p38 inhibitor compound. In still other embodiments, the expression of only one of these proteins is reduced following administration of a p38 inhibitor compound.

In some embodiments, the p38 inhibitor is not an ACE II inhibitor (e.g. the p38 inhibitor does not significantly reduce ACE II activity).

In one embodiment, atrial fibrosis in a subject is reduced following administration of a p38 inhibitor compound relative to prior to administration of the p38 inhibitor compound. In some embodiments, the atrial fibrosis is reduced by more than about 2%; more than about 5%; more than about 10%; or more than about 25%. In some aspects, a reduction of atrial fibrosis of a population of treated subjects may be measured by any reproducible means. For example, a reduction in atrial fibrosis may be measured by EP study, MRI, CAT scan, and the like.

The methods described herein may include identifying a subject in need of treatment. In a preferred embodiment, the methods include identifying a mammal in need of treatment. In a highly preferred embodiment, the methods include identifying a human in need of treatment. Identifying a subject in need of treatment may be accomplished by any means that indicates a subject who may benefit from treatment. For example, identifying a subject in need of treatment may occur by clinical diagnosis, laboratory testing, or any other means known to one of skill in the art, including any combination of means for identification. Examples include, but are not limited to, listening to the subject's heartbeat, taking the subject's pulse, an electrocardiogram (EKG), a Holter monitor or other similar device for the continuous recording of the heart rhythm, a patient-activated or automatically-triggered event recorder or other similar device whereby the subject's heart rhythm is recorded at the onset of symptoms, echocardiography, ultrasound, transesophageal echocardiography (TEE), electrophysiologic (EP) studies, and the like. In addition, high blood pressure and signs of heart failure may be ascertained during a physical examination of the subject. Blood tests may be performed to detect abnormalities in blood oxygen and carbon dioxide levels, electrolytes, and thyroid hormone levels. Chest x-rays, CAT scans, and MRI may reveal enlargement of the heart, heart failure, and other diseases of the lung. Exercise treadmill testing may be used to detect severe coronary artery disease

As described elsewhere herein, the compounds described herein may be formulated in pharmaceutical compositions, if desired, and may be administered by any route that permits treatment of the disease or condition. A preferred route of administration is oral administration. Administration may take the form of single dose administration, or the compound of the embodiments may be administered over a period of time, either in divided doses or in a continuous-release formulation or administration method (e.g., a pump). However the compounds of the embodiments are administered to the subject, the amounts of compound administered and the route of administration chosen should be selected to permit efficacious treatment of the disease condition.

The methods of the embodiments also include the use of a compound or compounds as described herein together with one or more additional therapeutic agents for the treatment of disease conditions. Additional therapeutic agents for the treatment of AF are well-known in the art and include, for example, digoxin, beta blockers (atenolol, metoprolol, propranolol), amiodarone, disopyramide, calcium antagonists (verapamil, diltiazam), sotalol, flecamide, procainamide, quinidine and propafenone. Thus, for example, the combination of active ingredients may, be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

In addition, embodiments of the invention include the use of a compound or compounds as described herein together with one or more AF therapies. AF therapies are well-known in the art, and include, for example, anti-arrhythmic therapy, electrical cardioversion, surgical procedures, such as the Maze procedure, ablation, radiofrequency energy, atrial pacemakers, and the like. Thus, for example, the compounds described herein may be administered before, during or after one or more AF therapies.

### p38 Inhibitors

A “p38 inhibitor” is a compound that inhibits (e.g., reduces) the activity of p38, e.g., inhibits the activity of a p38 MAPK. The inhibitory effects of a compound on the activity of p38 may be measured by various methods well-known to a skilled artisan. For example, the inhibitory effects may be measured by measuring the level of inhibition of lipopolysaccharide (LPS)-stimulated cytokine production (Lee et al. 1988 Int J Immunopharmacol 10:835-843; Lee et al. 1993 Ann NY Acad Sci 696:149-170; Lee et al. 1994 Nature 372:739-746; Lee et al. 1999 Pharmacol Ther 82:389-397).

Pirfenidone (5-methyl-1-phenyl-2-(1H)-pyridone) is a known compound and its pharmacological effects are disclosed, for example, in Japanese Patent Application KOKAI (Laid-Open) Nos. 87677/1974 and 1284338/1976. U.S. Pat. Nos. 3,839,346; 3,974,281; 4,042,699; and 4,052,509; each of which is hereby incorporated by reference in its entirety, describe methods of manufacture of 5-methyl-1-phenyl-2-(1H)-pyridone and its use as an anti-inflammatory agent.

In addition to pirfenidone, the p38 inhibitor compounds described below (including the compounds of Genera Ia-c, Subgenera II-V and Genus VI) are useful in the methods described herein.

The term “alkyl” used herein refers to a monovalent straight or branched chain radical of from one to ten carbon atoms, including, but not limited to, methyl, ethyl, n-propyl, isopropyl, n-butyl, isobutyl, tert-butyl, n-hexyl, and the like.

The term “alkenyl” used herein refers to a monovalent straight or branched chain radical of from two to ten carbon atoms containing a carbon double bond including, but not limited to, 1-propenyl, 2-propenyl, 2-methyl-1-propenyl, 1-butenyl, 2-butenyl, and the like.

The term “halo” used herein refers to fluoro, chloro, bromo, or iodo.

The term “haloalkyl” used herein refers to one or more halo groups appended to an alkyl radical.

The term “nitroalkyl” used herein refers to one or more nitro groups appended to an alkyl radical.

The term “thioalkyl” used herein refers to one or more thio groups appended to an alkyl radical.

The term “hydroxyalkyl” used herein refers to one or more hydroxy groups appended to an alkyl radical.

The term “alkoxy” used herein refers to straight or branched chain alkyl radical covalently bonded to the parent molecule through an —O— linkage. Examples of alkoxy groups include, but are limited to, methoxy, ethoxy, propoxy, isopropoxy, butoxy, n-butoxy, sec-butoxy, t-butoxy and the like.

The term “alkoxyalkyl” used herein refers to one or more alkoxy groups appended to an alkyl radical.

The term “carboxy” used herein refers to —COOH.

The term “alkoxycarbonyl” refers to —(CO)—O-alkyl. Examples of alkoxycarbonyl groups include, but are limited to, methoxycarbonyl group, ethoxycarbonyl group, propoxycarbonyl group, and the like.

Carbohydrates are polyhydroxy aldehydes or ketones, or substances that yield such compounds upon hydrolysis. Carbohydrates comprise the elements carbon (C), hydrogen (H) and oxygen (O) with a ratio of hydrogen twice that of carbon and oxygen.

In their basic form, carbohydrates are simple sugars or monosaccharides. These simple sugars may combine with each other to form more complex carbohydrates. The combination of two simple sugars is a disaccharide. Carbohydrates consisting of two to ten simple sugars are called oligosaccharides, and those with a larger number are called polysaccharides.

The term “uronide” refers to a monosaccharide having a carboxyl group (—COOH) on the carbon that is not part of the ring. The uronide name retains the root of the monosaccharide, but the -ose sugar suffix is changed to -uronide. For example, the structure of glucuronide corresponds to glucose.

As used herein, a radical indicates species with a single, unpaired electron such that the species containing the radical may be covalently bonded to another species. Hence, in this context, a radical is not necessarily a free radical. Rather, a radical indicates a specific portion of a larger molecule. The term “radical” may be used interchangeably with the term “group.”

As used herein, a substituted group is derived from the unsubstituted parent structure in which there has been an exchange of one or more hydrogen atoms for another atom or group. When substituted, the substituent group(s) is (are) one or more group(s) individually and independently selected from alkyl, cycloalkyl, aryl, fused aryl, heterocyclyl, heteroaryl, hydroxy, alkoxy, aryloxy, mercapto, alkylthio, arylthio, cyano, halo, carbonyl, thiocarbonyl, alkoxycarbonyl, nitro, silyl, trihalomethanesulfonyl, trifluoromethyl, and amino, including mono- and di-substituted amino groups, and the protected derivatives thereof. The protecting groups that may form the protective derivatives of the above substituents are known to those of skill in the art and may be found in references such as Greene and Wuts Protective Groups in Organic Synthesis; John Wiley and Sons: New York, 1999. Wherever a substituent is described as “optionally substituted” that substituent may be substituted with the above substituents.

The term “purified” refers to a compound which has been separated from other compounds such that it comprises at least 95% of the measured substance when assayed.

Asymmetric carbon atoms may be present in the compounds described herein. All such isomers, including diastereomers and enantiomers, as well as the mixtures thereof are intended to be included in the scope of the recited compound. In certain cases, compounds may exist in tautomeric forms. All tautomeric forms are intended to be included in the scope of the recited compound. Likewise, when compounds contain an alkenyl or alkenylene group, there exists the possibility of cis- and trans-isomeric forms of the compounds. Both cis- and trans-isomers, as well as the mixtures of cis- and trans-isomers, are contemplated. Thus, reference herein to a compound includes all of the aforementioned isomeric forms unless the context clearly dictates otherwise.

Various forms are useful in the methods described herein, including polymorphs, solvates, hydrates, conformers, salts, and prodrug derivatives. A polymorph is a composition having the same chemical formula, but a different structure. A solvate is a composition formed by solvation (the combination of solvent molecules with molecules or ions of the solute). A hydrate is a compound formed by an incorporation of water. A conformer is a structure that is a conformational isomer. Conformational isomerism is the phenomenon of molecules with the same structural formula but different conformations (conformers) of atoms about a rotating bond. Salts of compounds may be prepared by methods known to those skilled in the art. For example, salts of compounds may be prepared by reacting the appropriate base or acid with a stoichiometric equivalent of the compound. A prodrug is a compound that undergoes biotransformation (chemical conversion) before exhibiting its pharmacological effects. For example, a prodrug may thus be viewed as a drug containing specialized protective groups used in a transient manner to alter or to eliminate undesirable properties in the parent molecule. Thus, reference herein to a compound includes all of the aforementioned forms unless the context clearly dictates otherwise.

The compounds described below are useful in the methods described herein. In an embodiment, a compound of Genera Ia-c, Subgenera II-V and/or Genus VI as described below exhibits an IC₅₀ in the range of about 100 μM to about 1000 μM for inhibition of p38 MAPK.

An embodiment provides a family of compounds represented by the following genus (Genus Ia):

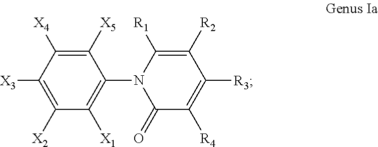

wherein

R₁, R₂, R₃, and R₄ are independently selected from the group consisting of H, alkyl, substituted alkyl, alkenyl, haloalkyl, nitroalkyl, thioalkyl, hydroxyalkyl, alkoxy, phenyl, substituted phenyl, halo, hydroxyl, alkoxyalkyl, carboxy, alkoxycarbonyl, CO-uronide, CO-monosaccharide, CO-oligosaccharide, and CO-polysaccharide; and

X₁, X₂, X₃, X₄, and X₅ are independently selected from the group consisting of H, halo, alkoxy, and hydroxy.

Another embodiment provides a family of compounds represented by the following genus (Genus Ib):

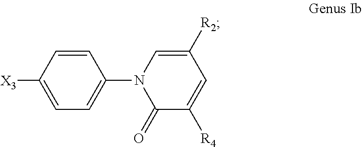

wherein

X₃ is selected from the group consisting of H, halogen, and OH;

R₂ is selected from the group consisting of H, C₁-C₆ alkyl, substituted C₁-C₆ alkyl, C₁-C₆ hydroxyalkyl, alkoxyalkyl, carboxy, C₁-C₆ alkoxycarbonyl, CO-uronide, CO-monosaccharide, CO-oligosaccharide, and CO-polysaccharide; and

R₄ is selected from the group consisting of H, halogen, and OH.

Another embodiment provides a family of compounds represented by the following genus (Genus Ic):

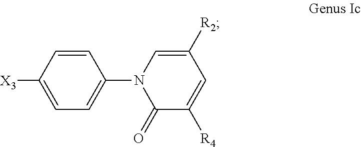

wherein

X₃ is selected from the group consisting of H, F, and OH;

R₂ is selected from the group consisting of H, CF₃, CH₂OH, COOH, CO-Glucoronide, CH₃, and CH₂OCH₃; and

R₄ is selected from the group consisting of H and OH;

with the proviso that when R₄ and X₃ are H, R₂ is not CH₃.

Another embodiment provides a family of compounds represented by the following subgenus (Subgenus II):

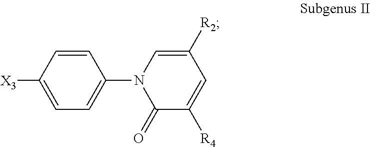

wherein

X₃ is selected from the group consisting of H and OH;

R₂ is selected from the group consisting of H, CH₂OH, COOH, CO-Glucoronide, CH₃, and CH₂OCH₃; and

R₄ is selected from the group consisting of H and OH.

Another embodiment provides a family of compounds represented by the following subgenus (Subgenus III):

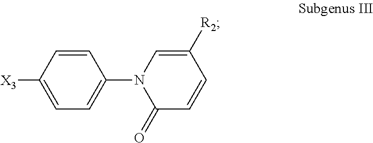

wherein

X₃ is selected from the group consisting of H, F, and OH; and

R₂ is selected from the group consisting of H and CF₃.

Another embodiment provides a family of compounds represented by the following subgenus (Subgenus IV):

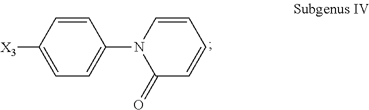

wherein X₃ is selected from the group consisting of H, halo, alkoxy, OH, alkyl, substituted alkyl, alkenyl, haloalkyl, nitroalkyl, thioalkyl, hydroxyalkyl, phenyl, substituted phenyl, alkoxyalkyl, carboxy, alkoxycarbonyl, CO-uronide, CO-monosaccharide, CO-oligosaccharide, and CO-polysaccharide.

Another embodiment provides a family of compounds represented by the following subgenus (Subgenus V):

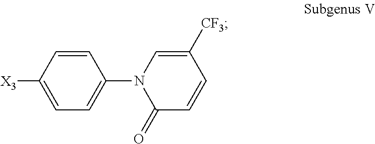

wherein X₃ is selected from the group consisting of H, halo, alkoxy, OH, alkyl, substituted alkyl, alkenyl, haloalkyl, nitroalkyl, thioalkyl, hydroxyalkyl, phenyl, substituted phenyl, alkoxyalkyl, carboxy, alkoxycarbonyl, CO-uronide, CO-monosaccharide, CO-oligosaccharide, and CO-polysaccharide.

Another embodiment provides a family of compounds represented by the following genus (Genus VI):

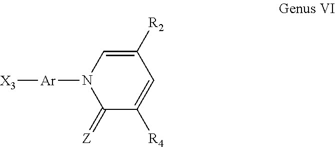

wherein

Ar is pyridinyl or phenyl;

Z is O or S; and

X₃ is H, F, Cl, OH, or OCH₃;

R₂ is methyl, C(═O)H, C(═O)CH₃, C(═O)O-glucosyl, fluoromethyl, difluoromethyl, trifluoromethyl, methylmethoxyl, methylhydroxyl, or phenyl; and

R₄ is H or hydroxyl;

with the proviso that when R₂ is trifluoromethyl, Z is O, R₄ is H and Ar is phenyl, the phenyl is not solely substituted at the 4′ position by H, F, or OH.

The Genus VI includes the families of compounds represented by the Subgenus VIa and the Subgenus VIb:

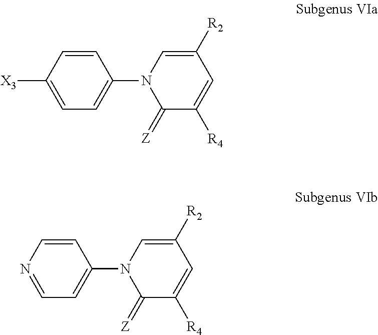

wherein Z, X₃, R₂ and R₄ are defined as in Genus VI. It will be recognized that the phenyl ring in the structure represented by Subgenus VIa is substituted by X₃ at the 4′ position.

It will be recognized that a particular compound described herein may be a member of more than one of the various genera and subgenera described above. The compounds described herein are useful for treating and/or preventing AF in a subject. Exemplary compounds of Genera Ia-c, Subgenera II-V and Genus VI that are useful for treating and/or preventing AF in a subject are set forth in Table 1 below. Compounds I-6 are examples of compounds of Subgenus II. Compounds 7-12 are examples of compounds of Subgenus III. Compound 13 is pirfenidone, an example of a compound of Subgenus II. Compounds 14-23 are examples of compounds of Genus VI.

TABLE 1

| Compound | |
|---|---|
| Number | Compound |
| 1 | 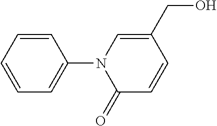 |
| 2 | 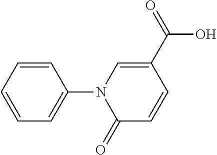 |
| 3 | 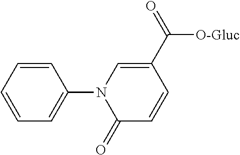 |
| 4 | 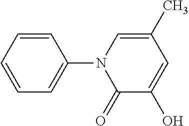 |
| 5 | 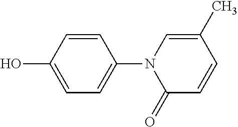 |
| 6 | 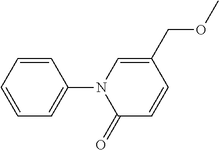 |
| 7 | 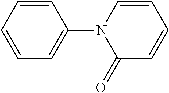 |
| 8 | 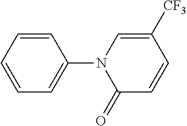 |
| 9 | 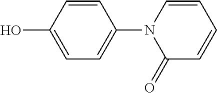 |
| 10 | 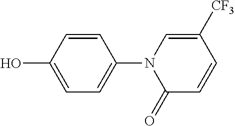 |
| 11 | 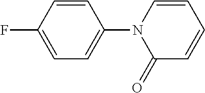 |
| 12 | 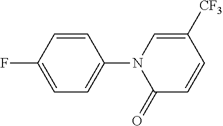 |
| 13 | 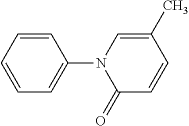 |
| 14 | 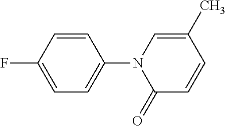 |
| 15 | 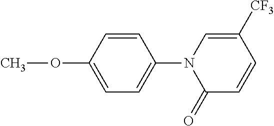 |
| 16 | 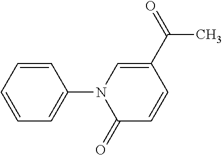 |
| 17 | 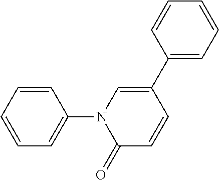 |
| 18 | 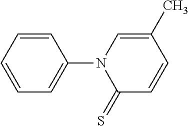 |
| 19 | 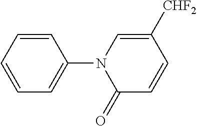 |
| 20 | 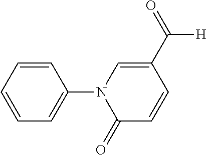 |
| 21 | 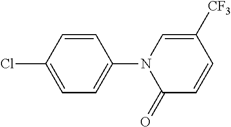 |
| 22 | 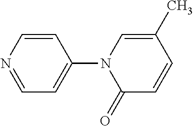 |
| 23 | 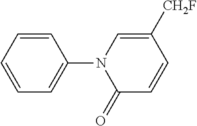 |

In preferred embodiments, purified compounds represented by Genera Ia-c, Subgenera II-V and/or Genus VI have a purity of about 96% or greater, more preferably about 98% or greater, by weight based on total weight of the composition that comprises the purified compound.

Compounds of Genera Ia-c, Subgenera II-V and/or Genus VI may be synthesized by using various conventional reactions known in the art. Examples of syntheses include the following, designated Synthetic Scheme 1.

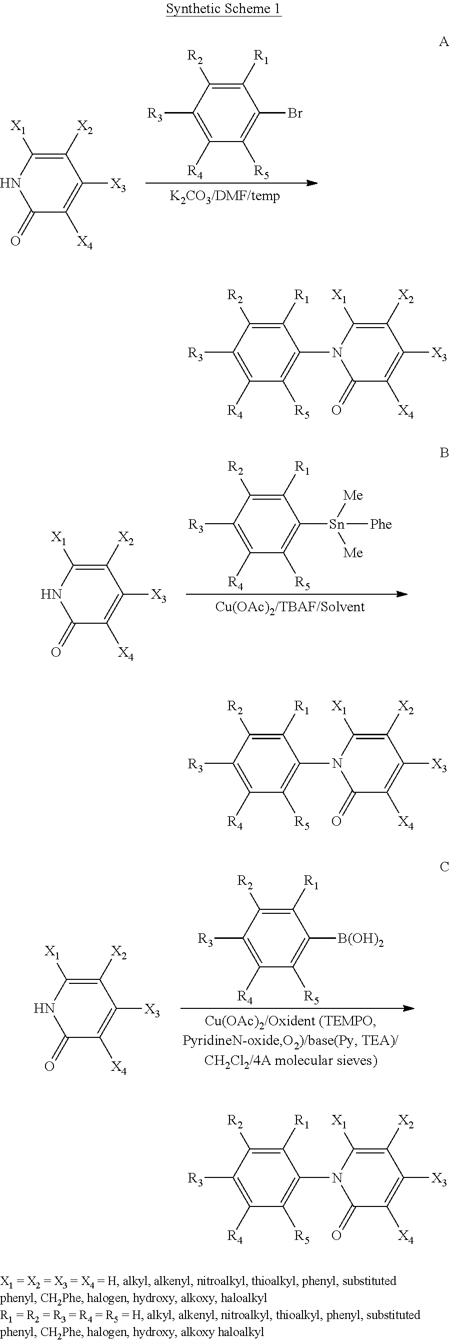

Compounds of Genera Ia-c, Subgenera II-V and/or Genus VI may also be synthesized by any conventional reactions known in the art based on the known synthetic schemes for pirfenidone, such as disclosed in U.S. Pat. Nos. 3,839,346; 3,974,281; 4,042,699; and 4,052,509.

Starting materials described herein are available commercially, are known, or may be prepared by methods known in the art. Additionally, starting materials not described herein are available commercially, are known, or may be prepared by methods known in the art.

Starting materials may have the appropriate substituents to ultimately give desired products with the corresponding substituents. Alternatively, substituents may be added at any point of synthesis to ultimately give desired products with the corresponding substituents.

Synthetic Scheme 1 shows methods that may be used to prepare the compounds of Genera Ia-c, Subgenera II-V and/or Genus VI. One skilled in the art will appreciate that a number of different synthetic reaction schemes may be used to synthesize the compounds of Genera Ia-c, Subgenera II-V and/or Genus VI. Further, one skilled in the art will understand that a number of different solvents, coupling agents, and reaction conditions may be used in the syntheses reactions to yield comparable results.

One skilled in the art will appreciate variations in the sequence and, further, will recognize variations in the appropriate reaction conditions from the analogous reactions shown or otherwise known which may be appropriately used in the processes above to make the compounds of Genera Ia-c, Subgenera II-V and/or Genus VI.

In the processes described herein for the preparation of the compounds of compounds of Genera Ia-c, Subgenera II-V and/or Genus VI, the use of protective groups is generally well recognized by one skilled in the art of organic chemistry, and accordingly the use of appropriate protecting groups may in some cases be implied by the processes of the schemes herein, although such groups may not be expressly illustrated. Introduction and removal of such suitable protecting groups are well known in the art of organic chemistry; see for example, T. W. Greene, “Protective Groups in Organic Synthesis”, Wiley (New York), 1999. The products of the reactions described herein may be isolated by conventional means such as extraction, distillation, chromatography, and the like.

The salts, e.g., pharmaceutically acceptable salts, of the compounds of Genera Ia-c, Subgenera II-V and/or Genus VI may be prepared by reacting the appropriate base or acid with a stoichiometric equivalent of the compounds. Similarly, pharmaceutically acceptable derivatives (e.g., esters), metabolites, hydrates, solvates and prodrugs of the compounds of Genera Ia-c, Subgenera II-V and/or Genus VI may be prepared by methods generally known to those skilled in the art. Thus, another embodiment provides compounds that are prodrugs of an active compound. In general, a prodrug is a compound which is metabolized in vivo (e.g., by a metabolic transformation such as deamination, dealkylation, de-esterification, and the like) to provide an active compound. A “pharmaceutically acceptable prodrug” means a compound which is, within the scope of sound medical judgment, suitable for pharmaceutical use in a patient without undue toxicity, irritation, allergic response, and the like, and effective for the intended use, including a pharmaceutically acceptable ester as well as a zwitterionic form, where possible, of the compounds of the embodiments. Examples of pharmaceutically-acceptable prodrug types are described in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

The compounds and compositions described herein may also include metabolites. As used herein, the term “metabolite” means a product of metabolism of a compound of the embodiments or a pharmaceutically acceptable salt, analog, or derivative thereof, that exhibits a similar activity in vitro or in vivo to a compound of the embodiments. The compounds and compositions described herein may also include hydrates and solvates. As used herein, the term “solvate” refers to a complex formed by a solute (herein, a compound of Genera Ia-c, Subgenera II-V and/or Genus VI) and a solvent. Such solvents for the purpose of the embodiments preferably should not interfere with the biological activity of the solute. Solvents may be, by way of example, water, ethanol, or acetic acid. In view of the foregoing, reference herein to a particular compound or genus of compounds will be understood to include the various forms described above, including pharmaceutically acceptable salts, esters, prodrugs, metabolites and solvates thereof.

### Pharmaceutical Compositions

While it is possible for the compounds useful in the methods described herein to be administered alone, it may be preferable to formulate the compounds as pharmaceutical compositions. As such, in yet another aspect, pharmaceutical compositions useful in the methods of the invention are provided. More particularly, the pharmaceutical compositions described herein may be useful, inter alia, for treating or preventing AF. A pharmaceutical composition is any composition that may be administered in vitro or in vivo or both to a subject in order to treat or ameliorate a condition. In a preferred embodiment, a pharmaceutical composition may be administered in vivo. A mammal includes any mammal, such as by way of non-limiting example, cattle, pigs, sheep, goats, horses, camels, buffalo, cats, dogs, rats, mice, and humans. A highly preferred subject mammal is a human.

In an embodiment, the pharmaceutical compositions may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8. More particularly, the pharmaceutical compositions may comprise a therapeutically or prophylactically effective amount of at least one compound as described herein, together with one or more pharmaceutically acceptable excipients. Optionally, the pharmaceutical compositions may comprise a combination of the compounds described herein, or may include a second active ingredient useful in the treatment or prevention of bacterial infection (e.g., anti-bacterial or anti-microbial agents).

Formulations, e.g., for parenteral or oral administration, are most typically solids, liquid solutions, emulsions or suspensions, while inhalable formulations for pulmonary administration are generally liquids or powders, with powder formulations being generally preferred. A preferred pharmaceutical composition may also be formulated as a lyophilized solid that is reconstituted with a physiologically compatible solvent prior to administration. Alternative pharmaceutical compositions may be formulated as syrups, creams, ointments, tablets, capsules and the like.

The term “pharmaceutically acceptable excipient” refers to an excipient for administration of a pharmaceutical agent, such as the compounds described herein. The term refers to any pharmaceutical excipient that may be administered without undue toxicity. Pharmaceutically acceptable excipients may include, for example, inactive ingredients such as disintegrators, binders, fillers, and lubricants used in formulating pharmaceutical products.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

Disintegrator include, for example, agar-agar, algins, calcium carbonate, carboxmethylcellulose, cellulose, clays, colloid silicon dioxide, croscarmellose sodium, crospovidone, gums, magnesium aluminium silicate, methylcellulose, polacrilin potassium, sodium alginate, low substituted hydroxypropylcellulose, and cross-linked polyvinylpyrrolidone hydroxypropylcellulose, sodium starch glycolate, and starch.

Binders include, for example, microcrystalline cellulose, hydroxymethyl cellulose, hydroxypropylcellulose, and polyvinylpyrrolidone.

Fillers include, for example, calcium carbonate, calcium phosphate, dibasic calcium phosphate, tribasic calcium sulfate, calcium carboxymethylcellulose, cellulose, dextrin derivatives, dextrin, dextrose, fructose, lactitol, lactose, magnesium carbonate, magnesium oxide, maltitol, maltodextrins, maltose, sorbitol, starch, sucrose, sugar, and xylitol.

Lubricants include, for example, agar, calcium stearate, ethyl oleate, ethyl laureate, glycerin, glyceryl palmitostearate, hydrogenated vegetable oil, magnesium oxide, magnesium stearate, mannitol, poloxamer, glycols, sodium benzoate, sodium lauryl sulfate, sodium stearyl, sorbitol, stearic acid, talc, and zinc stearate.

The pharmaceutical compositions described herein may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed. To those skilled in the pharmaceutical research and manufacturing, it is generally known that tablet formulations permit generous additions of inactive ingredients including excipients and coating substances, and a high percentage of fillers. However, the addition of inactive ingredients may limit the amount of active ingredients carried in each tablet.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil. Capsules may allow for inclusion of a larger amount of binders, instead of fillers as used more in tablets. In one embodiment, by weight, 2-10% of the capsule is disintegrator, 2-30% is binder, 2-30% is filler, and 0.3-0.8% is lubricant. A multitude of substances may be suitably included as disintegrator, binder, filler, and lubricant. One example is to use magnesium stearate as lubricant, microcrystalline cellulose as binder, and croscarmellose as disintegrator. In one embodiment, the capsule formulation further includes povidone. By weight povidone may constitute 1-4% of the capsule. The capsule shell may be made of hard gelatin in one embodiment. The shell may be clear or opaque, white or with color in various embodiments. In one embodiment, the capsule is size 1. Other sizes may be adopted in alternative embodiments.

In another embodiment, pharmaceutical compositions may be formulated as suspensions comprising a compound of the embodiments in admixture with at least one pharmaceutically acceptable excipient suitable for the manufacture of a suspension.

In yet another embodiment, pharmaceutical compositions may be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcellulose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions may also be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions may be in the form of a sterile injectable preparation, such as a sterile injectable aqueous emulsion or oleaginous suspension. This emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids such as oleic acid may likewise be used in the preparation of injectables.

To obtain a stable water-soluble dose form of a pharmaceutical composition, a pharmaceutically acceptable salt of a compound described herein may be dissolved in an aqueous solution of an organic or inorganic acid, such as 0.3 M solution of succinic acid, or more preferably, citric acid. If a soluble salt form is not available, the compound may be dissolved in a suitable co-solvent or combination of co-solvents. Examples of suitable co-solvents include alcohol, propylene glycol, polyethylene glycol 300, polysorbate 80, glycerin and the like in concentrations ranging from about 0 to about 60% of the total volume. In one embodiment, the active compound is dissolved in DMSO and diluted with water.

The pharmaceutical composition may also be in the form of a solution of a salt form of the active ingredient in an appropriate aqueous vehicle, such as water or isotonic saline or dextrose solution. Also contemplated are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In a preferred embodiment, the compounds described herein may be formulated for oral administration in a lipid-based formulation suitable for low solubility compounds. Lipid-based formulations may generally enhance the oral bioavailability of such compounds.

As such, a preferred pharmaceutical composition comprises a therapeutically or prophylactically effective amount of a compound described herein, together with at least one pharmaceutically acceptable excipient selected from the group consisting of—medium chain fatty acids or propylene glycol esters thereof (e.g., propylene glycol esters of edible fatty acids such as caprylic and capric fatty acids) and pharmaceutically acceptable surfactants such as polyoxyl 40 hydrogenated castor oil.

In an alternative preferred embodiment, cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. A particularly preferred cyclodextrin solubility enhancer is hydroxypropyl-o-cyclodextrin (BPBC), which may be added to any of the above-described compositions to further improve the aqueous solubility characteristics of the compounds of the embodiments. In one embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of the compound of the embodiments in the composition.

A pharmaceutical composition preferably contains a total amount of the active ingredient(s) sufficient to achieve an intended therapeutic effect. More specifically, in some embodiments, the pharmaceutical composition contains a therapeutically effective amount (e.g., an amount of a p38 inhibitor compound that is effective in the prevention or treatment of AF). The total amounts of the compound that may be combined with the carrier materials to produce a unitary dosing form will vary depending upon the host treated and the particular mode of administration. Preferably, the compositions are formulated so that a dose of between 0.01 to 100 mg/kg body weight/day of a p38 inhibitor compound is administered to a subject receiving the compositions.

It is to be understood that the description, specific examples and data, while indicating exemplary embodiments, are given by way of illustration and are not intended to limit the various embodiments of the present disclosure. All references cited herein for any reason, are specifically and entirely incorporated by reference. Various changes and modifications within the present disclosure will become apparent to the skilled artisan from the description and data contained herein, and thus are considered part of the various embodiments of this disclosure. Individual embodiments may specifically include or exclude any such alternatives.

### EXAMPLES

The effects of pirfenidone (PFD) on arrhythmogenic atrial remodeling and AF vulnerability in canines with ventricular tachypacing (VTP)-induced congestive heart failure (CHF) were assessed as described below. The results of the study demonstrate that VTP-induced CHF is associated with marked arrhythmogenic LA remodeling with a significant increase in AF vulnerability, and PFD treatment resulted in a significant reduction in both LA remodeling and AF vulnerability.

### Example 1

### Animal Model

Briefly, 15 adult mongrel canines (weight 20 to 32 kgs) were divided into 3 groups (n=5 in each group) as follows: Group 1: Normal; Group 2: CHF canines not treated with PFD (or CHF); and Group 3: CHF canines treated with PFD (or CHF+PFD). The Normal canines did not undergo pacemaker implantation and were not given the PFD. The CHF and CHF+PFD canines underwent placement of a permanent single-chamber pacemaker with the pacing lead positioned in the right ventricular apex followed by radiofrequency catheter ablation of the AV junction to create complete heart block. Canines in the CHF and CHF+PFD groups underwent 3 weeks of VTP at a rate of 220 bpm. Oral PFD (800 mg three times a day) (InterMune, Brisbane, Calif.) was started 2 days prior to the initiation of VTP and was given for the full duration of the pacing period.

On follow-up, the animals underwent open-chest electrophysiological (EP) and mapping studies, as described in Verheule S, et al., Circulation 107:2615-22 (2003) and Sih H J, et al. J Am Coll Cardiol. 36:924-31 (2000). Atrial tissue samples were processed for histologic and staining studies.

### Statistical Analysis

Data variables were checked for normality and equality of variances (Kolmogoroz-Smirnov and Levene's tests). Comparisons of multiple group differences were performed using ANOVA with post-hoc Bonferroni correction. In the case that the data variable (AF duration) was not normally distributed with equal variances, the Kruskal-Wallis test was used. All results were presented as mean±SD, and p<0.05 was deemed statistically significant. Data analysis was carried out with the SPSS 13.0 software package.

### Example 2

### Monitoring of the CHF Model

The CHF and CHF+PFD groups underwent transesophageal echocardiography at the time of pacemaker implantation and at follow-up. Canines in the paced groups underwent weekly transthoracic echocardiography, weekly ECG monitoring to ensure right ventricular capture, and weekly physical examinations. CHF was established by clinical signs, such as, lethargy, peripheral edema, and mucous membrane color changes. Left atrial (LA) size was determined by measuring the LA area by planimetry from 2-D echocardiographic images during diastole from the 2-chamber views. Left ventricular (LV) systolic function was determined by measuring LV fractional shortening at the level of the papillary muscle. Two repeated measurements were made for LA area and LV fractional shortening and the mean value was used for analyses.

### Left Ventricular Function, Left Atrial Dilatation

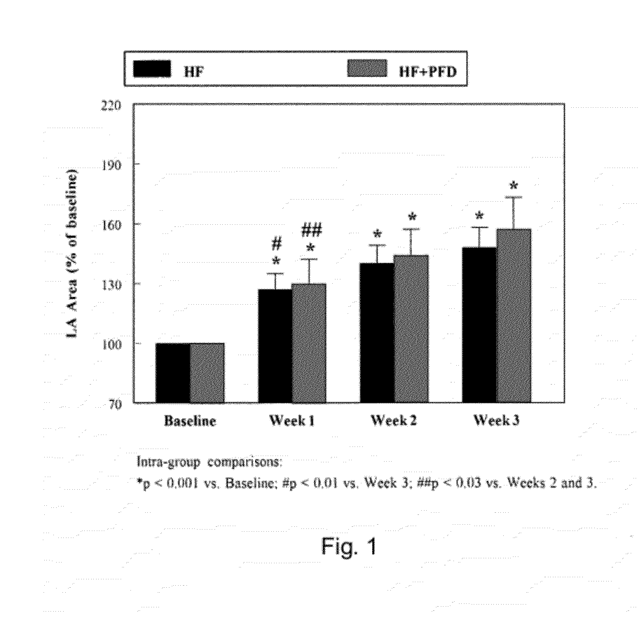

LV fractional shortening after 3 weeks of VTP was markedly reduced for both the CHF (−63±7%, p<0.001) and CHF+PFD (−69±8%, p<0.001) canines when compared with baseline. The inter-group baseline and weekly LV fractional shortening measurements for the CHF and CHF+PFD groups were similar. For both groups, LA area (FIG. 1) was significantly increased after 1 week of VTP and this increase was progressive over the 3 weeks of VTP. The increase in LA area from baseline at each weekly time point was similar between the 2 paced groups. CHF signs did not appear to be different between the paced groups.

### Example 3

### Electrophysiological Study

During the follow-up EP study, each animal was anesthetized with isoflurane and mechanically ventilated. The pacemaker rate was set at 80 bpm at twice diastolic threshold for the entire EP study. The chest was opened with a midline sternotomy. A pericardial cradle was created, and 4 custom-made, epicardial, high-density plaques (left atrial free wall (LAFW); left atrial Bachmann's bundle (LABB); right atrial free wall (RAFW); right atrial Bachmann's bundle (RABB)) were placed over the atria (512 electrodes with an inter-electrode distance of 2.5 mm), similar to the setup described in Verheule S, et al., Circulation 107:2615-22 (2003) and Sih H J, et al. J Am Coll Cardiol. 36:924-31 (2000). Unipolar electrode signals were acquired (sampling rate 2 kHz) and stored with the UnEmap mapping system (University of Auckland, New Zealand). Electrode pairs on the epicardial plaque were used for bipolar stimulation at twice diastolic threshold. Effective refractory periods (ERPs) were measured at 12 atrial sites (6 in LA, 6 in RA) using the single extrastimulus protocol (S₁S₂) at an 8-beat drive train basic cycle lengths (BCLs) of 200, 300, and 400 ms. During stimulation of the contralateral Bachmann's bundle, conduction velocity (CV) was calculated between pairs of plaque electrodes perpendicular to the activation wavefront with a custom-written software. Verheule S, et al., Am J Physiol Heart Circ Physiol. 2004; 287:H634-44; and Bayly P V, et al., IEEE Trans Biomed Eng. 1998; 45:563-71. Used as a marker of conduction heterogeneity (Verheule S, et al., Am J Physiol Heart Circ Physiol. 2004; 287:H634-44; Bayly P V, et al., IEEE Trans Biomed Eng. 1998; 45:563-71; and Ausma J, et al., Circulation. 1997; 96:3157-63), the phase difference (ms/mm) was defined as the average difference in activation time between a plaque electrode from all of its neighboring electrodes normalized by the inter-electrode distance. Frequency histograms were constructed for the phase differences within an atrial region. The histograms were summarized as the median phase (P₅₀), and the 5^{th} and 95^{th} percentile phase, or P₅ and P₉₅ of the distribution, respectively. Two measures were derived to quantify conduction heterogeneity: 1) absolute conduction heterogeneity, defined as P₉₅-P₅ (P₉₅₋₅), and 2) conduction heterogeneity index, defined as the absolute conduction heterogeneity normalized by the median phase, or P₉₅₋₅/P₅₀.

AF inducibility was assessed by both the single-extrastimulus protocol (as above) and a burst pacing protocol which consisted of pacing at one LA site and one RA site. A total of 16 burst stimulations were carried out for each animal with each atrial site receiving 8 burst pacings (4 for a duration of 6 seconds and 4 for 12 seconds) at a CL of 50 ms and a stimulus output of 0.5 V+twice diastolic threshold. AF was considered sustained if the induced episode lasted >30 minutes at which time the longest AF duration was taken as 3600 seconds and used for analysis.

### Atrial Fibrillation Vulnerability

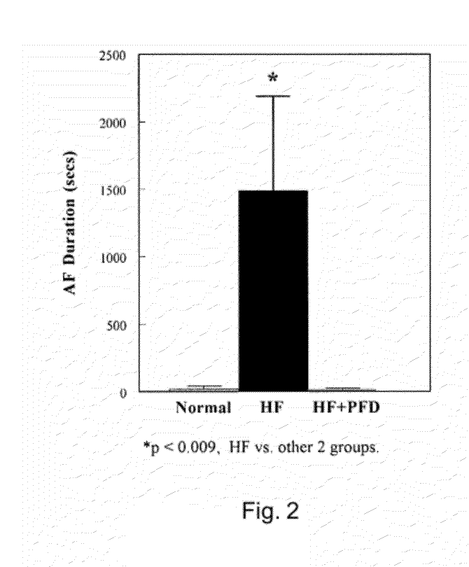

In open-chest experiments, sustained AF was only observed in the untreated CHF canines (4/5, p<0.007). VTP-induced CHF resulted in a significant increase in mean AF duration, from 16±25 secs in the Normal group to 1488±698 secs (p<0.009) (FIG. 2). PFD treatment resulted in a significant reduction in mean AF duration to 12±13 secs (p<0.009 vs. CHF) that was similar to that found in the Normal group.

### Open-Chest Electrophysiologic Studies

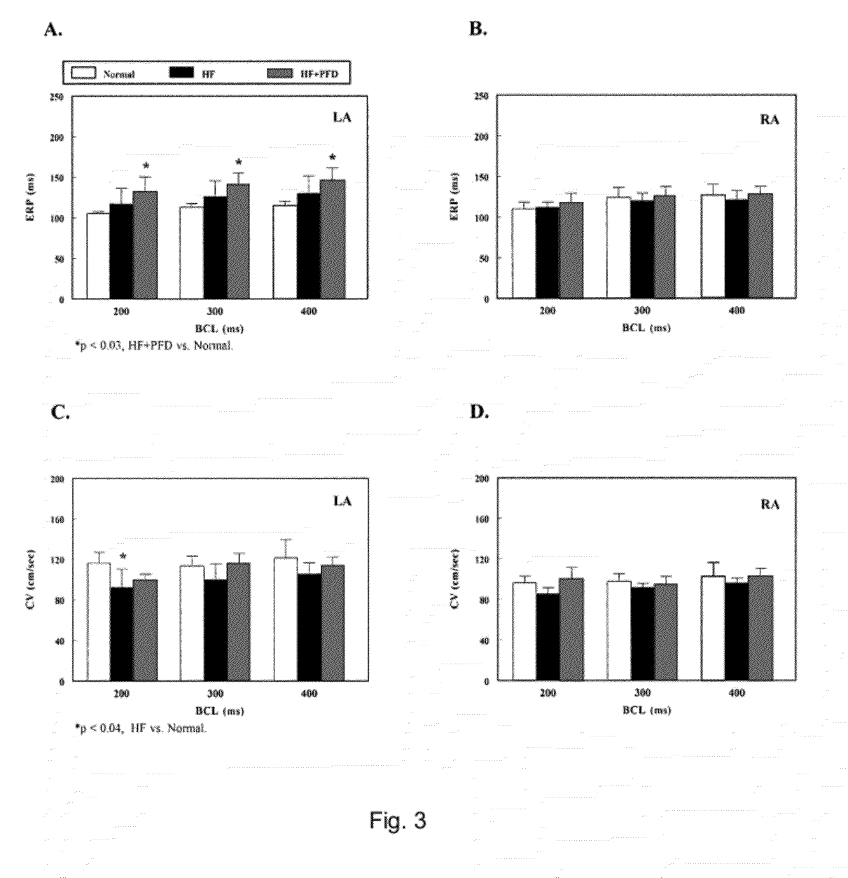

Shown in FIGS. 3A and 3B are the LA and RA ERPs, respectively, for the study groups at 3 pacing BCLs (200, 300, and 400 ms). VTP-induced CHF resulted in a trend toward longer ERPs in the LA compared with the Normals (p=NS). Treatment with PFD resulted in further lengthening in LA ERPs compared with untreated canines (p=NS) and Normal canines (p<0.03 for all BCLs). RA ERPs were similar among all groups. Shown in FIGS. 3C and 3D are the LA and RA CVs, respectively, for the study groups at 3 pacing BCLs. Compared with the LA CVs in the Normal group, LA CVs in canines with VTP-induced CHF were decreased at all BCLs, reaching statistical significant at the BCL of 200 ms (p<0.04). Treatment with PFD resulted in a non-statistically significant increase in LA CVs compared with the untreated group. CVs in the RA were similar among the three groups.

### Conduction Heterogeneity

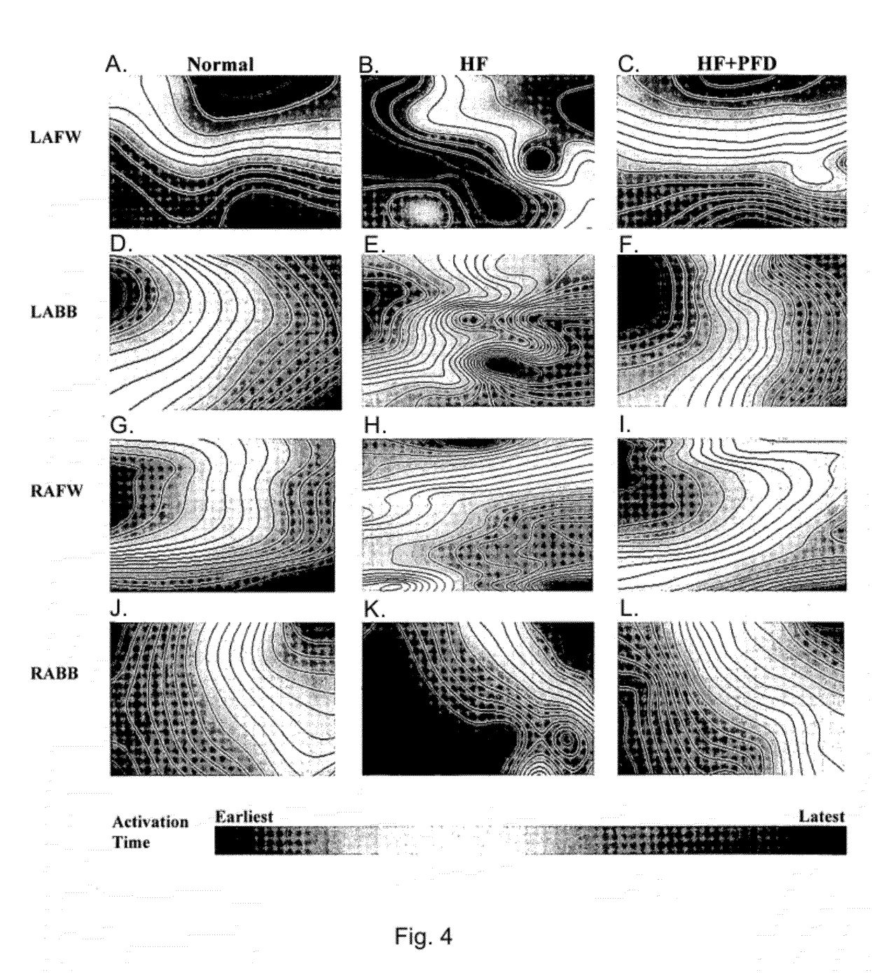

Shown in FIG. 4 are comparisons of the isochronal activation maps for each of the 4 atrial plaques at a pacing CL of 300 ms. Atrial conduction was more heterogeneous (more discrete areas of slow conduction) in the CHF group compared with the Normal group, and this local conduction heterogeneity was less with PFD treatment.

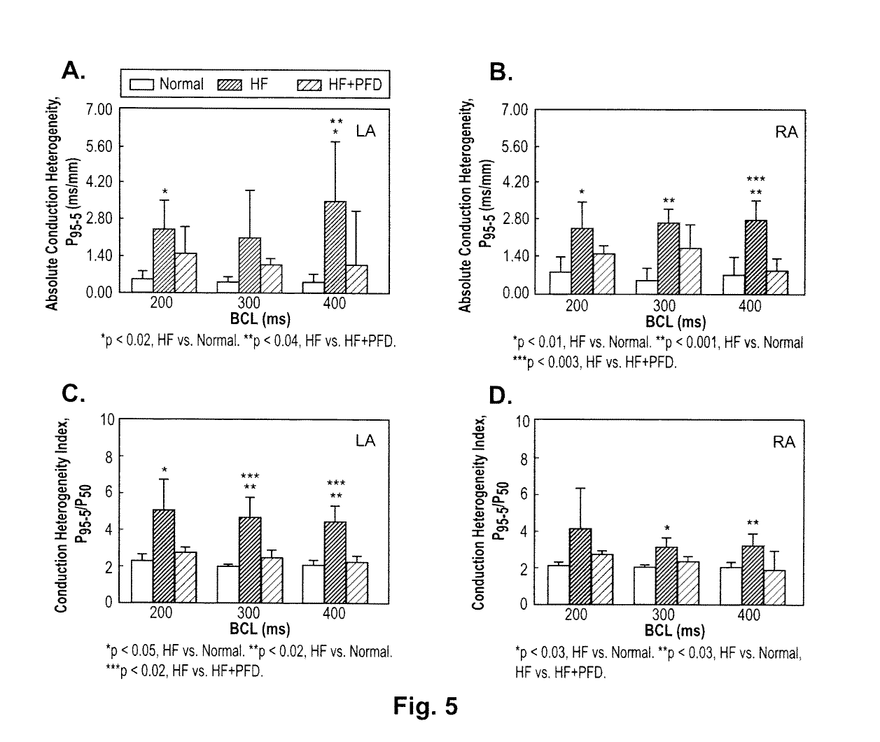

Atrial conduction heterogeneity was also analyzed with phase delay maps and derivation of absolute conduction heterogeneity and conduction heterogeneity index, plotted in FIGS. 5A-D. VTP-induced CHF resulted in an increase in both measures of conduction heterogeneity in the LA at all BCLs compared with Normals (p<0.02 at 300 and 400 ms for absolute heterogeneity; p<0.05 at 200 ms and p<0.02 at 300, 400 ms for heterogeneity index). Treatment with PFD resulted in a reduction in both measures of conduction heterogeneity in the LA at all BCLs (p<0.04 at 400 ms for absolute heterogeneity; p<0.02 at 300 and 400 ms for heterogeneity index). As for conduction heterogeneity in the RA, there was an increase in both measures of conduction heterogeneity at all BCLs in the CHF canines compared with Normals, and a decrease in both measures with PFD treatment (p<0.003 at 400 ms for absolute heterogeneity and for heterogeneity index). The median phase (not shown) was similar for all groups at all BCLs.

### Example 4

### Histologic Studies

At the conclusion of the EP study, the animals were euthanized. Atrial tissue samples were fixed in 10% neutral buffered formalin. The samples were processed, embedded in paraffin, and sectioned into 4- to 5-μm-thick sections. The sections were stained in either H&E, Masson's trichrome, or Sirius red. Section images were digitized using a Spot Camera (Diagnostics Instruments, Sterling Heights, Mich.). To quantify fibrosis, the red pixel content of digitized images (Sirius red-stained) was measured relative to the total tissue area (red and green pixels) with the Adobe Photoshop 7.0 software package. Areas containing blood vessels and perivascular interstitial cells were excluded from fibrosis quantification. Atrial tissue samples were frozen in liquid nitrogen and homogenized in solubilization buffer.

### Histologic Findings

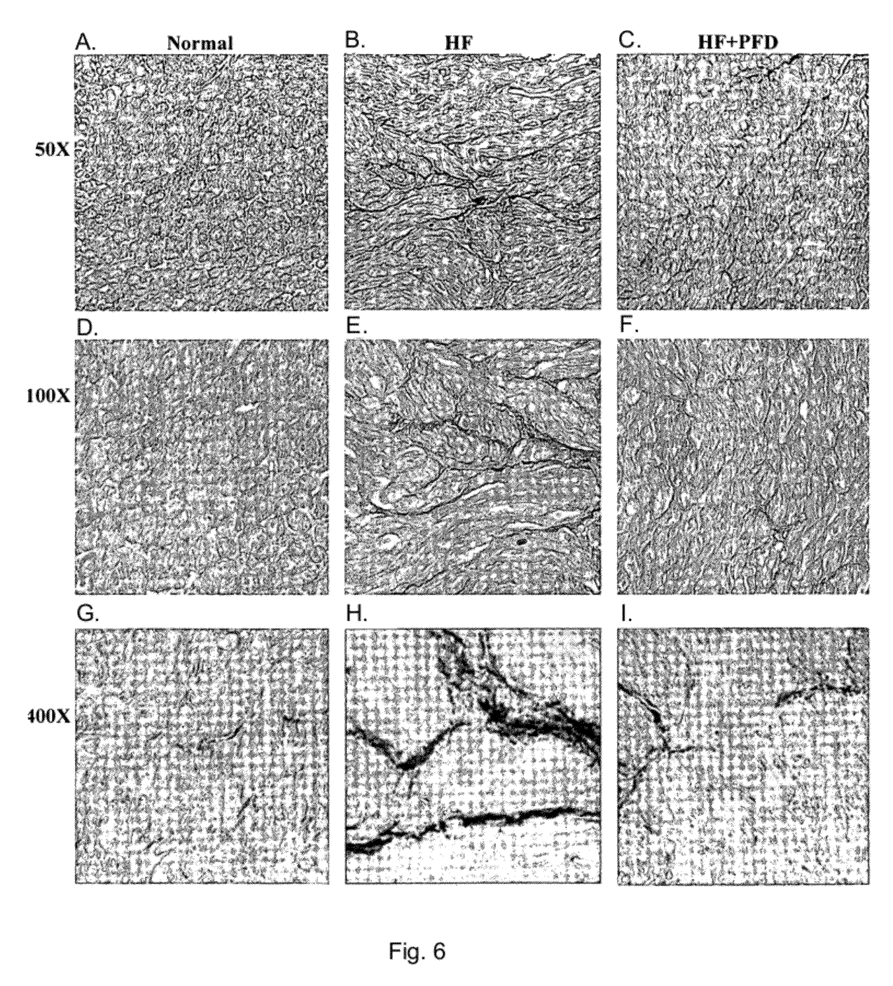

Representative LA sections stained with Sirius red are shown in FIG. 6. The LA of canines not subjected to VTP appeared normal. However, LA sections in untreated CHF canines had extensive interstitial fibrosis. Furthermore, myocyte hypertrophy and cell loss were more prominent in the untreated CHF group. Treatment with PFD resulted in significant attenuation in interstitial fibrosis. Histologic alterations were also seen in the RA (not shown) although they were much more extensive in the LA.

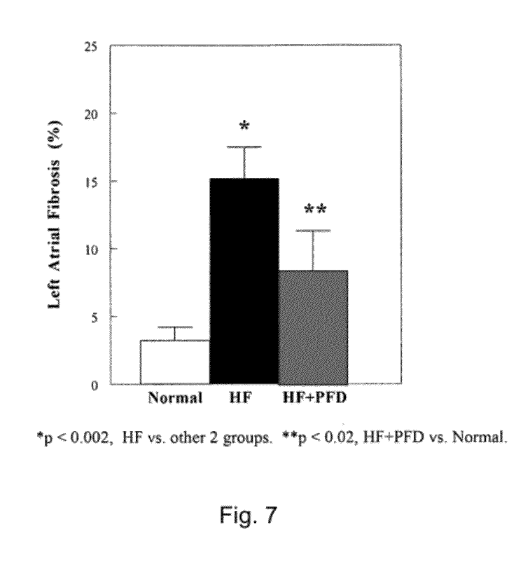

Fibrosis quantification was performed from the Sirius red-stained specimens (FIG. 7). There was a significant increase in percentage LA fibrosis in untreated CHF canines compared with Normals (15.4±2.3% vs. 3.2±1.0%, p<0.002). PFD treatment resulted in a significant reduction in percentage LA fibrosis (8.3±3.0%, p<0.002 vs. CHF group), although it was still greater than that found in Normals (p<0.02).

### Example 5

Expression of MAPks in atrial tissue was evaluated using Western Blot analysis. Briefly, atrial tissue specimen containing an equal amount of total protein (10 μg) was electrophoresed on a 4-20% Tris-glycine gel and then transferred onto a nitrocellulose filter. Non-specific binding sites were blocked with 4% BSA, and the filter was incubated with diluted antibody and a matched secondary antibody (all antibodies were obtained from Chemicon, Temecular, Calif.). Protein bands were analyzed with an enhanced chemiluminescence detection method using horseradish peroxidase, based on the recommendations from the manufacturer (NEN Life Science, Boston, Mass.).

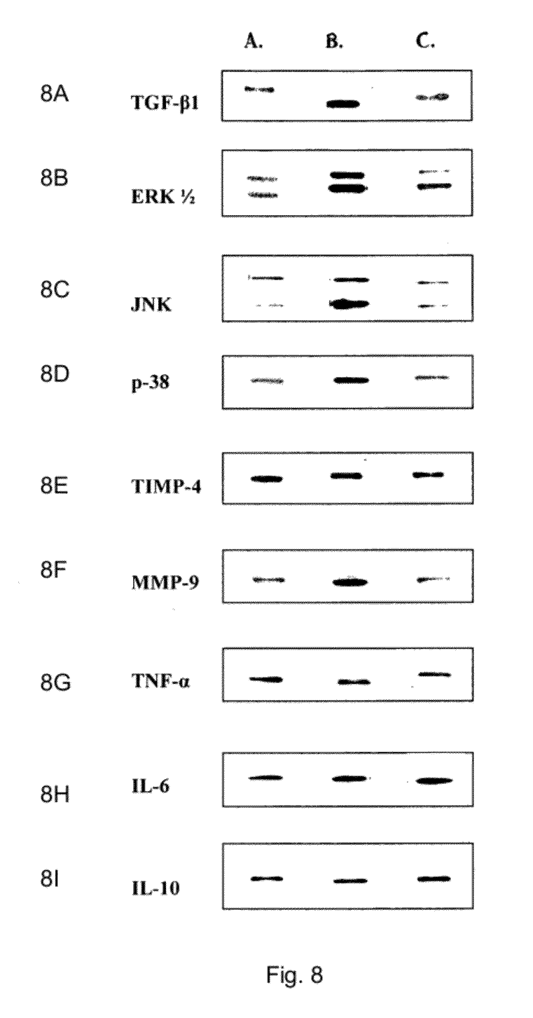

FIG. 8 shows the Western immunoblot results for transforming growth-factor (TGF)-β1, total extracellular signal-regulated protein kinase (ERK), total c-Jun N-terminal kinase (JNK), total p-38, tissue inhibitor of metalloproteinase (TIMP)-4, matrix metalloproteinase (MMP)-9, TNF-α, IL-6, and IL-10. VTP-induced CHF resulted in an upregulation in the expression of TGF-β1, ERK, JNK, p-38, and MMP-9, while PFD treatment resulted in a downregulation of their expression. Expression of TIMP-4, TNF-α, IL-6, and IL-10 were unchanged in the all 3 groups.

The renin-angiotensin system plays an important role in formation of myocardial fibrosis in various structural heart disease. Weber K T, et al., Cardiovasc Res. 1993; 27:341-8; Brilla C G, et al., Circ Res. 1990; 67:1355-64; Tan L B, et al., J Hypertens Suppl. 1992; 10:S31-4; Urata H, et al., J Clin Invest. 1993; 91:1269-81. While circulating angiotensin II (Ang II) is an important promoter of connective tissue formation (Weber K T, et al. Int J Biochem Cell Biol. 1999; 31:395-403), the effects of Ang II are mediated by mitogen-activated protein kinases (MAPKs) on the tissue level. Yano M, et al., Circ Res. 1998; 83:752-60; Sugden P H, Clerk A., J Mol. Med. 1998; 76:725-46. In patients with atrial fibrosis and A F, Goette et al. have found elevated Ang II concentration with increased ERK activation. Goette A, et al., J Am Coll Cardiol. 2000; 35:1669-77. Furthermore, Li et al. have reported that in a canine model, VTP-induced CHF resulted in an increase in Ang II concentration and expression of MAPK subfamilies ERK, c-Jun, and p38 (total and phosphorylated). Li D, et al., Circulation. 2001; 104:2608-14. Li et al. also found that treatment with an ACE inhibitor (enalapril) led to a reduction of Ang II concentration and ERK activation with less arrhythmogenic atrial remodeling. In the instant study, 3-weeks of VTP resulted in an increase in expression of total ERK, c-Jun, and p38, all of which were reduced with PFD treatment.

Atrial extracellular matrix homeostasis is regulated by a delicate balance of MMPs and their endogenous inhibitors (TIMPs), with TIMP-4 the most cardiospecific. Li Y Y, et al., Circulation. 1998; 98:1728-34; Thomas C V, et al., Circulation. 1998; 97:1708-15; Li H, et al., Cardiovasc Res. 2000; 46:298-306; Greene J, et al.; J Biol. Chem. 1996; 271:30375-80. MMPs mediate the degradation of extracellular matrix proteins and their upregulation may lead to cardiomyopathy. Thomas C V, et al., Circulation. 1998; 97:1708-15; Spinale F G, et al., Circ Res. 1999; 85:364-76. Nakano et al. has found that the expression of the active form of MMP-9 (88 kDa) was significantly increased in AF patients. Nakano Y, et al., J Am Coll Cardiol. 2004; 43:818-25. In the instant study, MMP-9 expression was increased with VTP-induced CHF and reduced with PFD treatment. TIMP-4 expression, on the other hand, was not markedly changed among the 3 study groups. These results are consistent with those of Boixel et al. who found that progressive heart failure and LA fibrosis in a rat model is associated with upregulation of MMPs but not TIMPs. Boixel C, et al., J Am Coll Cardiol. 2003; 42:336-44.

Previous work has shown that overexpression of the potent pro-fibrotic mediator TGF-β1 in transgenic mice resulted in an increase in atrial interstitial fibrosis, conduction heterogeneity, and AF vulnerability. Verheule S, et al., Circ Res. 2004; 94:1458-65. PFD has been reported to significantly reduce expression of TGF-β1 in a animal models of lung fibrosis (Iyer S N, et al., J Pharmacol Exp Ther. 1999; 291:367-73), .hepatic fibrosis (Garcia L, et al., J Hepatol. 2002; 37:797-805), and renal tubulointerstitial fibrosis (Shihab F S, et al., Am J Transplant. 2002; 2:111-9). In the instant study, VTP resulted in a marked increase in TGF-β1 expression, which was reduced with PFD treatment.

It has also been reported that inflammation may play a prominent role in the promotion of AF. Chung M K, et al., Circulation. 2001; 104:2886-91; Aviles R J, et al., Circulation. 2003; 108:3006-10; Ishii Y, et al., Circulation. 2005; 111:2881-8. Although, recently, Goette et al. have found that while atria obtain from AF patients during open heart surgery had increased calpain enzymatic activity, no activation of tissue cytokines was observed. Goette A, et al., Am J Physiol Heart Circ Physiol. 2002; 283:H264-72. The subjects in that study had prolonged, chronic AF with mean arrhythmia duration of 47 months, and the associated inflammation may have diminished significantly over time. In the instant study, inflammatory markers, TNF-α, IL-6, and IL-10, were not markedly different in the 3 study groups.

### Example 6

The distribution of gap junction proteins connexin 43 (Cx43) and connexin 40 (Cx40) in atrial tissue was also studied. Atrial specimens were incubated with mouse monoclonal antibody against Cx40 and rabbit polyclonal antibody against Cx43 (Dako) overnight at 4° C. Subsequently, incubation with FITC-labeled goat anti-rabbit (for Cx43) and Texas Red-labeled donkey anti-mouse (for Cx40) antibodies (Jackson ImmunoResearch Laboratories, West Grove, Pa.) was performed. The specimens were processed and analyzed with fluorescent microscopy.

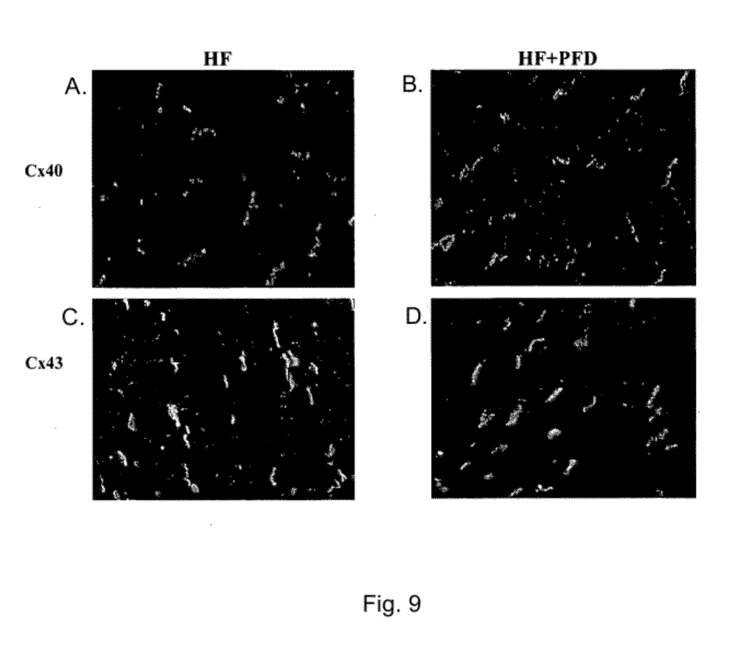

Distribution of Cx43 and Cx40 in LA in the CHF and CHF+PFD groups was also studied (FIG. 9). Spatial distribution of both of these gap junction proteins did not appear markedly different in the treated and untreated groups.

These results suggest that PFD attenuates atrial fibrosis and AF vulnerability predominantly via its antifibrotic effects, without apparent alteration in spatial distribution Cx40 and Cx43.

### Discussion

After 3 weeks of VTP, canines in this study developed significant LA fibrosis, LV dysfunction, and LA dilatation, similar to those reported by others. Li D, et al., Circulation 1999; 100:87-95; and Shinagawa K, et al., Circulation. 2002; 105:2672-8. Although canines that were treated with PFD had similar CHF severity as their untreated counterparts, the treated group had a significant reduction in LA fibrosis and AF vulnerability. Notable electrophysiologic changes with PFD treatment included a trend toward an increase in LA ERP's and CV's, which may be due to improved cell-to-cell coupling because of less interstitial fibrosis.

### Example 7

Two adult mongrel canines (weight 20 to 32 kgs) with heart failure produced by 4 weeks of rapid ventricular pacing as described above were evaluated for AF inducibility following PFD treatment.

Briefly, pacemakers were turned off at 4 weeks and PFD started for 3 weeks. A follow-up study for AF inducibility after 3 weeks of PFD treatment was performed as described above. In both animals AF was not found.

### Example 8

Patients diagnosed with AF participate in a double-blind, placebo controlled, randomized study to provide insight into the treatment of AF using p38 inhibitor compounds. The diagnosis of AF is confirmed by EKG. Patients are randomly assigned into p38 inhibitor compound or placebo using a modified permuted-block randomization method. Patients receive oral tablets (p38 inhibitor or placebo) at a dose of 400 mg three times a day for the course of the study 3 weeks.

The AF burden, amount of time spent in AF and duration of AF episodes, in patients is monitored throughout the course of the study using automatically-triggered event recording devices. For patients receiving p38 inhibitor, AF is reversed or AF burden is significantly reduced as compared to prior to treatment. The amount of time spent in AF is reduced on average by 95% compared to prior to treatment. For patients who experience an AF episode, the duration of the episode is reduced on average by 95%. For patients receiving placebo, the amount of time spent in AF and the duration of AD episodes are largely unchanged compared to prior to treatment.

### Example 9

Patients having just underwent a cardiac operation participate in a double-blind, placebo controlled, randomized study to provide insight into the prevention of AF in high-risk patients using p38 inhibitor compounds. Patients are randomly assigned into p38 inhibitor compound or placebo using a modified permuted-block randomization method. Patients receive oral tablets (p38 inhibitor or placebo) at a dose of 100 mg three times a day for the course of the study 3 months.

Patients are monitored throughout the course of the study using automatically-triggered event recording devices. Of patients receiving p38 inhibitor, less than 5% experience AF. However, AF occurs in 50% of patients receiving placebo.

### Example 10

Preparation of 1-(4-hydroxyphenyl)-5-(trifloromethyl)-2-pyridone (Compound 10): A mixture of 5-(trifloromethyl)-2(1H)-pyridone (815.5 mg, 5 mmol), 4-iodoanisole (2.34 g, 10 mmol), CuI (952 mg, 5 mmol), K₂CO₃ (691 mg, 5 mmol) and DMF (5 ml) was heated at 135° C. overnight. The reaction mixture was diluted with 10% ammonia (15 ml) and extracted with ethyl acetate. The organic extract was washed with saturated sodium chloride, dried over magnesium sulfate and evaporated. Column chromatography purification (30% ethyl acetate-hexane) afforded 526 mg (39.2%) of 1-(4-methoxyphenyl)-5-(trifloromethyl)-2-pyridone. This compound (268.2 mg, 1 mmol) was treated with 1M BBr₃ solution in dichloromethane (DCM, 2 ml) in DCM (5 ml) for 2 hours at 0° C. Reaction mixture was diluted with DCM and washed 3 times with water. Organic phase was dried over sodium sulfate and evaporated. The residue was separated by column chromatography (20% ethyl acetate-DCM) to afford the title compound as an off-white solid, 226 mg (89%). The ¹H NMR spectra was consistent with the structure of Compound 10.

### Example 11

Preparation of 1-phenyl-5-acetyl-2-pyridone (Compound 16): 2-methoxy-5-acetyl pyridine (1.51 g, 10 mmol) was treated with 6N HCl at 100° C. for 5 hours. The reaction mixture was neutralized with sodium hydroxide to pH 7 and then extracted several times with DCM. Organic layer was dried over sodium sulfate, evaporated and the residue was crystallized from ethyl acetate to give 5-acetyl-2(1H)-pyridone as a white solid, 1.06 g (78%). This compound (685.7 mg, 5 mmol) was reacted with iodobenzene (0.84 ml, 7.5 mmol) in the presence of CuI (95 mg, 0.5 mmol) and K₂CO₃ (691 mg, 5 mmol) in DMF (5 ml) at 135° C. overnight. The reaction mixture was diluted with 10% ammonia (15 ml) and extracted with ethyl acetate. The organic extract was washed with saturated sodium chloride, dried over magnesium sulfate and evaporated. Column chromatography (10% ethyl acetate-DCM) afforded 407 mg (38%) of the target compound as a white solid. The ¹H NMR spectra was consistent with the structure of Compound 16.

### Example 12

Preparation of 1-(4-pyridinyl)-5-methyl-2-pyridone (Compound 22): Compound 22 was synthesized by condensation of 5-methyl-2(1H)-pyridone (327.4 mg, 3 mmol) with 4-bromopyridine hydrochloride (778 mg, 4 mmol) in the presence of CuI (60 mg, 0.3 mmol) and K₂CO₃ (1.36 g, 10 mmol) in DMF (3 ml) at 135° C. overnight. The reaction mixture was diluted with 10% ammonia (15 ml) and extracted with ethyl acetate. Organic extract was washed with saturated sodium chloride, dried over magnesium sulfate and evaporated. Column chromatography (5% MeOH-DCM) afforded 197 mg (35%) of the target compound as a yellowish solid. The ¹H NMR spectra was consistent with the structure of Compound 22.

### Example 13

Preparation of 1-phenyl-5-methyl-2-pyridinethione (Compound 18): 1-phenyl-5-methyl-2-pyridinone (555.7 mg, 3 mmol) was reacted with Lawesson's reagent (606.7 mg, 1.5 mmol) in toluene (5 ml) at 90° C. Reaction mixture was evaporated and the target compound was isolated by column chromatography (20-30% ethyl acetate-hexane) followed by crystallization from methyl-tert-butyl ether. Yield 403 mg (67%), yellow solid. The ¹H NMR spectra was consistent with the structure of Compound 18.

### Example 14.1

Characterization of Compound Efficacy in a Transgenic Mouse Model of Atrial Fibrosis/Fibrillation

Experiments employ a strain of transgenic mice designed to express a TGF-β variant under the control of a myosin heavy chain (MHC) promoter. The TGF-β isoform expressed from this promoter carries a Cys-to-Ser mutation at position 33; this mutation prevents association into a latent complex which leads to increased levels of active TGF-β. Mice expressing this transgene develop selective atrial fibrosis (Nakajima et al Circ Res 2000: 86; 571-79) which has been shown to increase vulnerability for atrial fibrillation (Verheule et al Circ Res 2004: 94; 1458-65).

To assay the capacity of compounds to inhibit atrial fibrosis/fibrillation, transgenic mice in groups of eight are treated with either a p38 inhibitor compound or a vehicle control. Dosing of compound in feed can be initiated as soon as the animals are weaned (approximately post-natal day 21) or earlier if a p38 inhibitor compound is delivered by intraperitoneal injection. Two additional groups are normal mice (wild-type littermates) of the same strain that are treated with either vehicle or a p38 inhibitor compound. Dosing is continued for 1-4 months after which several end-points can be assessed as described in the examples below (Verhule et al Circulation Research 2004).

### Example 14.2

ECG and Open Chest Electrophysiology Studies

Methods to determine the effect of atrial fibrosis on surface ECG and open chest electrophysiology in this model have been described (Verheule et al 2004). The ECG of untreated transgenic mice will display a decreased P-wave amplitude. Treatment of transgenic mice with a p38 inhibitor compound is expected to restore P-wave amplitude to typical values. Transesophageal burst pacing of the left atrium is expected to induce atrial fibrillation in a subset of untreated transgenic mice. Treatment with a p38 inhibitor compound is expected to reduce either or both of the inducibility or duration of atrial fibrillation in transgenic mice.

### Example 14.3

Histologic Characterization of Fibrosis

Following electrophysiological studies, mice are sacrificed and fibrosis is characterized by histology in as described in Example 4. Briefly, hearts are mounted in freezing medium (Triangle Biomedical Science, Durham, N.C.), fixed with formalin and stained with either Sirius red/fast green or Masson trichrome. Previous studies have show that overexpression of TGF-β in this model leads to increased atrial fibrosis (Verheule et al Circulation Research 2004; Nakajima et al Circulation Research 200). Treatment of transgenic mice with a p38 inhibitor compound is expected to reduce the extent of fibrosis when compared to untreated transgenic mice.

### Example 14.4

Characterization of Levels of Fibrosis Associated Proteins

The levels of fibrosis-associated proteins of interest can be observed following sacrifice using methods described in the canine model described in Example 5. Examples of fibrosis-associated proteins of interest include but are not limited to TGF-β1 (human transgene expressed in mouse model), TGF-β1 (mouse), MMP-9, ERK-1/2, JNK, and p38 isoforms. As in the canine model described in Example 5, treatment with a p38 inhibitor compound is expected to modulate expression of one or more of these proteins. In some embodiments, treatment with a p38 inhibitor compound is expected to modulate expression of TNF-α (decreased expression) and/or TIMP-4 (increased expression).

What is claimed is:

1. A method of reducing or preventing atrial fibrillation (AF) in a mammalian subject in need thereof comprising administering to the subject an effective amount of pirfenidone to reduce or prevent atrial fibrillation.

2. The method of claim 1, comprising administering pirfenidone as an oral composition.

3. The method of claim 1, wherein the subject suffers from a heart disorder.

4. The method of claim 3, wherein the heart disorder is selected from the group consisting of congenital heart disease, coronary heart disease, myocardial infarction, congestive heart failure and combinations thereof.

5. The method of claim 1, wherein the subject is at risk for developing atrial fibrillation.

6. The method of claim 4, wherein the risk for developing atrial fibrillation is associated with the subject presenting a risk factor selected from the group consisting of increased age, high blood pressure, abnormal heart muscle function, disease of the mitral valve between the left and right ventricles, pericarditis, hyperthyroidism, overdose of thyroid medication, low amounts of oxygen in the blood, emphysema, asthma, chronic obstructive pulmonary disease, pulmonary embolism, physical or psychological stress, excessive alcohol intake, stimulant drug use, recent heart or lung surgery and combinations thereof.

7. The method of claim 1, wherein the mammalian subject is human.

8. The method of claim 1, wherein the amount of pirfenidone is effective to reduce the frequency of atrial fibrillation (AF) episodes or to reduce the subject's AF burden, relative to prior to administration of pirfenidone.

9. The method of claim 8, wherein the amount of pirfenidone is effective to reduce the frequency of atrial fibrillation (AF) episodes.

10. The method of claim 8, wherein the amount of pirfenidone is effective to reduce the subject's AF burden by reducing the subject's time spent in AF.

11. The method of claim 8, wherein the amount of pirfenidone is effective to reduce the subject's AF burden by reducing the duration of AF episodes.

12. The method of claim 8, wherein the amount of pirfenidone is effective to reduce both the subject's time spent in AF and the duration of AF episodes, relative to prior to administration of pirfenidone.

13. The method of claim 11, wherein the duration of AF episodes is reduced by at least 5% relative to prior to administration of pirfenidone.

14. The method of claim 1, wherein the subject is suffering from chronic AF.

15. The method of claim 1, wherein the subject is suffering from paroxysmal AF.

16. The method of claim 1, wherein the amount of pirfenidone is effective to delay the onset of or prevent chronic AF in said subject.

17. A method of reducing or preventing atrial fibrosis due to cardiac arrhythmia in a mammalian subject in need thereof comprising administering to the subject an effective amount of pirfenidone to reduce or prevent atrial fibrosis.

18. The method of claim 16, wherein the atrial fibrosis in the subject is reduced by at least 5% compared to a subject not administered pirfenidone.

19. The method of claim 18, wherein the mammalian subject is human.

20. The method of claim 17, wherein the amount of pirfenidone is effective to reduce or prevent atrial fibrillation (AF) in said subject.
